(19) United States
(12) Patent Application Publication
(10) Pub. No.: US 2026/0041409 A1
(43) Pub. Date: Feb. 12, 2026

(54) Oral Sampling Device

(71) Applicant: CaviSense Inc., Boston, MA (US)

(72) Inventors: Gili Naveh, Brookline, MA (US); Michael Natan, Cambridge, MA (US); Sabesh Kanagalingam, Billerica, MA (US)

(21) Appl. No.: 18/997,341

(22) PCT Filed: Jul. 21, 2023
(86) PCT No.: PCT/US2023/070780

§ 371 Date: Jan. 21, 2025

Related U.S. Application Data
(60) Provisional application No. 63/391,075, filed on Jul. 21, 2022.

(51) Int. Cl. A61B 10/00

(52) U.S. Cl. CPC A61B 10/0051; A61B 2010/0006

(57) ABSTRACT

An oral sampling device includes a handle configured to be gripped by a user, and an oral sampling tip extending outwardly from the handle. The oral sampling tip is sized to be inserted into an oral cavity of the user and provide a visual indication of biological data for fluids or solids located within the oral cavity.

### CROSS-REFERENCE TO RELATED APPLICATIONS

This application claims the benefit of U.S. Provisional Ser. No. 63/391,075 filed Jul. 21, 2022 entitled “Oral Sampling Device”, which is incorporated by reference herein in its entirety.

### TECHNICAL FIELD

The present disclosure generally relates to a handheld oral device, and in some embodiments, to a handheld oral sampling (e.g., for detecting interproximal cavities) and/or interproximal testing device.

### SUMMARY

In one embodiment there is an oral sampling device including a handle configured to be gripped by a user and an oral sampling tip extending outwardly from the handle, the oral sampling tip sized to be inserted into an oral cavity of the user and provide a visual indication of biological data for fluids or solids located within the oral cavity. In some embodiments, the handle includes a first portion and a second portion, the first portion having a width that is greater than a width of the second portion and the oral sampling tip is coupled to the second portion. In some embodiments, the oral sampling tip is detachably coupled to the handle. In some embodiments, a shaft extends outwardly from the second portion of the handle and is curved upwardly away from a bottom surface of the handle.

In some embodiments, the oral sampling device further includes a stopper coupled to a distal end of the shaft, the stopper having a diameter that is greater than a diameter of the shaft. In some embodiments, the visual indication is representative of a pH value of the fluids or solids located within the oral cavity. In some embodiments, the stopper is colored to match the visual indication of the oral sampling tip when the pH value of the fluids or solids within the oral cavity indicate a presence of dental caries. In some embodiments, the oral sampling tip includes a pointed distal tip.

In some embodiments, the oral sampling tip is comprised of an adsorbent material. In some embodiments, the oral sampling tip is comprised of paper. In some embodiments, the oral sampling tip has a maximum thickness of between about 0.025 mm to about 3.000 mm. In some embodiments, the handle includes a top surface and bottom surface each having a generally concave curvature. In some embodiments, the handle includes first and second side walls opposite one another and each extending between the bottom surface and the top surface, each of the first and second side walls includes a bottom portion extending or not extending downwardly from the bottom surface away from the top surface and a top portion extending upwardly from the top surface away from the bottom surface. In some embodiments, at least one of the top surface and bottom surface include a plurality of ribbed features extending along the respective top surface and bottom surface. In some embodiments, the oral sampling tip is configured to be inserted into an interproximal space in the oral cavity.

In another embodiment, there is a method of using an intraoral device, the method includes gripping a handle of the intraoral device, inserting an oral sampling tip coupled to the handle into an oral cavity of a user, and determining one or more dental conditions of the oral cavity based on an indication generated by the oral sampling tip relating to biological data of fluids or solids located within the oral cavity. In some embodiments, the method further includes detaching the oral sampling tip from the handle, coupling a second oral sampling tip to the handle, inserting the second oral sampling tip into the oral cavity of the user, and obtaining, via the second oral sampling tip, second biological data of the fluids or solids located within the oral cavity. In some embodiments, there is a shaft coupling the oral sampling tip to the handle, the shaft being detachably coupled to the handle and the oral sampling tip is fixedly coupled to the shaft, and the method further includes detaching the shaft and oral sampling tip fixedly coupled thereto from the handle, coupling a second shaft having a second oral sampling tip fixedly coupled thereto to the handle, inserting the second oral sampling tip into the oral cavity of the user, and obtaining, via the second oral sampling tip, second biological data of the fluids or solids located within the oral cavity.

In some embodiments, inserting the oral sampling tip into the oral cavity causes the oral sampling tip to change from a first color to a second color, and determining one or more dental conditions of the oral cavity includes determining, based on the change from the first color to the second color, a measure of acidity of the fluids or solids located within the oral cavity. In some embodiments, inserting the oral sampling tip into the oral cavity includes inserting the oral sampling tip into an interproximal space in the oral cavity.

In another embodiment there is a cavity detection device including a handle having a first portion and a second portion, the first portion having a width that is greater than a width of the second portion, a shaft coupled to and extending outwardly from the second portion of the handle, the shaft being curved away from a bottom surface of the handle, a stopper coupled to a distal end of the shaft, the stopper having a diameter that is greater than a diameter of the shaft, and a plurality of oral sampling tips each configured to be detachably coupled to the handle, and each being configured to be inserted into a pit or fissure of a tooth as well an interproximal space in an oral cavity of a user to measure a local pH value therein and change from a first color to a second color to provide a visual indication of the measured pH value to indicate a presence of dental caries.

### BRIEF DESCRIPTION OF THE DRAWINGS

The following detailed description of embodiments of the oral sampling device and method, will be better understood when read in conjunction with the appended drawings of an exemplary embodiment. It should be understood, however, that the invention is not limited to the precise arrangements and instrumentalities shown.

In the drawings:

FIG. 1 is a top perspective view of an oral sampling device in accordance with an exemplary embodiment of the present invention;

FIG. 2 is a bottom perspective view of the sampling device of FIG. 1;

FIG. 3 is a top elevational view of a portion of the oral sampling device of FIG. 1 including a sampling tip before and after an oral sampling;

FIG. 4 is a side cross-sectional view of the oral sampling device of FIG. 1;

FIGS. 5A-5D are views of a sampling tip for use with the oral sampling device of FIG. 1, in accordance with an embodiment of the present disclosure; and

FIG. 6 is a flowchart illustrating a method of using the oral sampling device in accordance with an exemplary embodiment of the present invention.

### DETAILED DESCRIPTION

With the recent developments of highly sensitive point of care diagnostics, saliva, which was regarded as a digestive fluid, is becoming a resource for monitoring and diagnosing a variety of diseases. Saliva is an easy to access body fluid that contains a wide range of biomarkers and molecules which are shown to be indicative of different localized and systemic diseases and health. Despite the great potential of salivary based diagnostics, there are several central challenges for its use. First, saliva is composed of 99% water, therefore the number of proteins and other biomarkers is relatively low, thus any salivary based diagnostics should be highly specific and sensitive. Second, there are different salivary glands in the mouth that are secreting saliva in different composition and concentrations. Moreover, salivary secretion is not constant throughout the day. Due to these reasons, salivary biomarker levels are constantly fluctuating and therefore multiple measurements are needed to generate a baseline. Third, in contrast to blood biomarkers, salivary molecules can respond to systemic changes but could also change due to local, dental diseases such as caries and periodontal disease. Furthermore, the dental conditions will often mask any systemic related changes. Although saliva can serve as an excellent diagnostic tool for dental diseases, it is rarely used as such due to, for example, limitation of localized repeated sampling. For example, tooth decay is caused by an acidic environment that is buffered by saliva. Salivary secretion may fluctuate during the day and therefore a single sampling may unlikely be representative of a continuous demineralization activity. Thus, to accurately assess and diagnose tooth demineralization and decay, may require sampling the same area of the patient's mouth multiple times spread throughout a day. However, such repeated sampling is difficult to perform in a dental office due to patients'limited availability and high costs associated with repeated visits.

As such, the oral sampling device and method of the present disclosure may enable a user to easily perform sampling and/or monitoring of biological data at the dental office, school setting, other health offices or hospitals and/or at home. Put another way, the oral sampling device and method thereof may enable localized measurements of a user's saliva and/or localized plaque and/or tooth surface to identify any localized dental related processes and establish a correct baseline for reliable diagnostics. In some embodiments, the oral sampling device of the present disclosure may be used to gather repeated samples to obtain biological data with any frequency, such as, but not limited to, once a day, twice a day, once every other day, once a week, or to gather a single sample to obtain biological data.

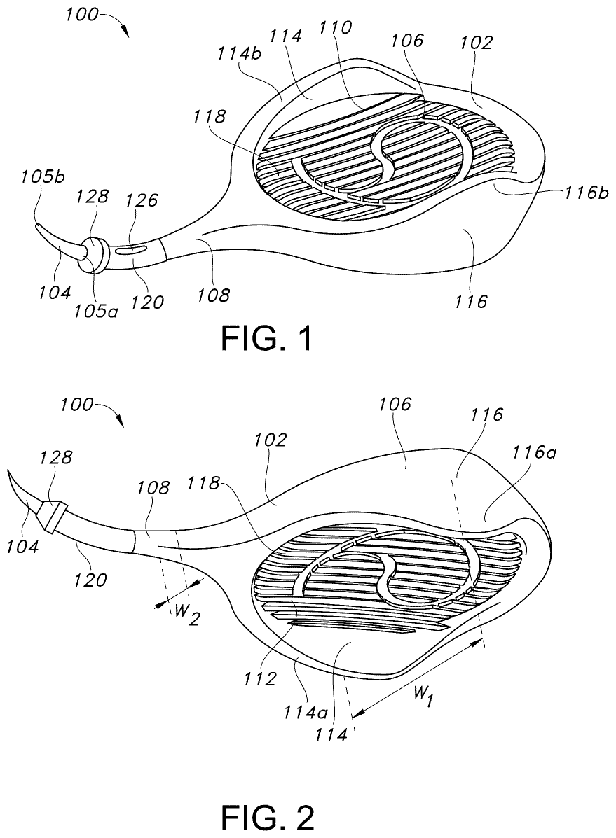

Referring to the drawings in detail, wherein like reference numerals indicate like elements throughout, there is shown in FIGS. 1-4 an oral sampling device, generally designated 100, also referred to as sampling device 100, in accordance with an exemplary embodiment of the present invention. The oral sampling device 100 may be configured to be easily gripped by a user (e.g., a dental healthcare practitioner, a patient, a care giver, or any other user) and be inserted into an oral cavity (e.g., a mouth) to gather biological data from the fluids or solids located within oral cavity. For example, the oral sampling device 100 may be configured to gather biological data from fluids or solids on or proximate a cheek, tooth, tongue, and/or an interproximal space (e.g., space between adjacent teeth). In some embodiments, the sampling device 100 may allow a user to easily and repeatedly gather biological data within the oral cavity to determine one or more dental conditions of the oral cavity.

Referring to FIGS. 1-2, the sampling device 100 includes a handle 102 and an oral sampling tip 104 coupled thereto. In some embodiments, the oral sampling tip 104 extends outwardly from the handle 102. The handle 102 may be sized and shaped to be easily gripped by a user. A user may refer to a dental healthcare practitioner (e.g., a dentist, a dental assistant), the patient themselves, a care giver, or any other user who operates the sampling device 100 to obtain biological data from within an oral cavity of a subject. A subject may include any persons for which the oral sampling is desired to be performed. As a non-limiting example, a subject may be a dental patient whom the sampling device 100 is to be used on and a user may be a dental healthcare practitioner operating the sampling device 100. A user may operate the sampling device 100 to obtain biological data from any and/or all surfaces of a tooth, gingival sulcus, within an interproximal space, a mouth, a cheek, and/or a tongue of a subject. In some instances, the user may be the subject as well or their caregiver (e.g., the user may operate the sampling device 100 to perform oral sampling on themselves).

The oral sampling tip 104 may be configured to obtain biological data from within the patient's mouth. For example, the oral sampling tip 104 may be configured to be inserted into an oral cavity, or mouth, of a patient to obtain biological data of fluids or solids located within one or more areas of the oral cavity (e.g., an interproximal space, the tongue, or cheeks). In some embodiments, the oral sampling tip 104 may be sized to extend at least partially into an interproximal space within the patient's oral cavity such that biological data from within the interproximal space may be obtained. For example, the sampling tip 104 may be sized to pass between the interproximal space between adjacent teeth such that biological data may be obtained therefrom. The sampling tip 104 may have a maximum thickness of less than 3.00 mm. In some embodiments, the sampling tip 104 has a maximum thickness of between about 0.025 mm to about 3.00 mm. In some embodiments, the oral sampling tip 104 is comprised of a paper having a generally uniform thickness within the range discussed previously. In other embodiments, the oral sampling tip 104 is generally conical in shape. The oral sampling tip 104 may have a thickness that tapers from a proximal end 105a towards the distal end 105b of the sampling tip 104. The distal tip 105b may be generally pointed and sized to be inserted into the interproximal space as well as pits and fissures of teeth and the gingival sulcus. In some embodiments, the oral sampling tip 104 is comprised of an adsorbent material. In some embodiments, the sampling tip 104 is comprised of one or more materials that are configured to react to the sampled fluids and/or solids within the tested space through direct contact or indirect fluid flow. A tested space may include a space, location, or area within the oral cavity for which oral sampling is desired, is currently being performed, or has been performed previously. In some embodiments, a tested space may be an interproximal space, a surface of a tooth, a gingival sulcus, or any other area within an oral cavity.

In some embodiments, one or more coatings may be applied to the oral sampling tip 104 such that biological data may be obtained via the one or more coatings. Sampling coatings and/or materials may include, for example, solutions (e.g., a liquid or paste or solid) that are configured to provide a visual indication relating to the biological data of sampled fluids or solids within the oral cavity. For example, the oral sampling tip 104 may be dipped into a sampling coating that is in a liquid, paste or solid state such that the exterior surface of the oral sampling tip 104 is coated by the sampling coating. The sampling coating may include a chemistry that is configured to change colors based on the pH level of fluids or solids. The sampling tip 104 may be dipped into the sampling coating such that the coating at least partially adheres to the sampling tip 104 and may be used to obtain biological data.

In some embodiments, only a portion of the sampling tip 104 may be coated. For example, only a distal portion (e.g., a portion extending from the distal end 105b partially towards the proximal end 105a) of the sampling tip 104 may immersed in the coating solution. In some embodiments, different portions of the sampling tip 104 may be coated in different sampling materials or coatings. For example, a first portion extending from the distal end 105b toward the proximal end 105a may be coated in a first sampling material configured to provide a visual indication relating to biological data of sampled fluids or solids. A second portion extending at least partially between the proximal end 105a and the distal end 105b may partially overlap the first portion and may be coated in a second sampling material configured to provide a visual indication relating to biological data of the sampled fluids or solids. A third portion, which may partially overlap the second portion, may be coated in a third sampling material configured to provide a visual indication relating to biological data of the sampled fluids or solids. In some embodiments, the first, second and/or third sampling materials may be configured to provide one or more different visual indications of the biological data obtained by sampling the fluids or solids within the oral cavity.

In some embodiments, one or more portions of the sampling tip 104 may be colored to provide a visual indication of the presence of one or more dental conditions. For example, the sampling tip 104 may have a first color (e.g., red, blue) that when visibly observable by a user indicates the presence of one or more dental and/or medical conditions. The sampling tip 104 may be coated, at least partially, in a sampling coating having a second color (e.g., green, yellow) that is different from the first color. The sampling coating may cover at least a portion of the sampling tip 104 such that the first color of the covered portion is obscured, and the second color is visible. The sampling tip 104 coated in the sampling coating may be inserted into the oral cavity to obtain biological data relating to the sampled fluids or solids. In the presence of one or more dental conditions (e.g., dental caries) the sampling coating may transition from the second color to be generally transparent or translucent such that the first color of the sampling tip is visible through the sampling coating. Alternatively, the sampling tip 104 may have a first color and it may be coated in a sampling coating that is initially transparent or translucent, such that the first color is observed. In the presence of one or more dental conditions (e.g., dental caries), the sample coating may transition from being transparent/translucent to a second color indicating the presence of the dental condition(s) and obscuring the first color of the sampling tip.

In some embodiments, the sampling tip 104 may be configured to deposit one or more materials onto, or in between, a subject's teeth. For example, the sampling tip 104 may include one or more non-adsorbent fibers to allow for the delivery of one or more pharmacological agents and/or dental materials, such as, but not limited to biological agents, proteins, restoring agents, remineralizing agents, sealants, and etchant agents to be deposited onto the subject's teeth. In some embodiments, the sampling tip 104 is comprised of a bio-manufactured, biocompatible, biodegradable, and/or edible material. In some embodiments, the sampling tip 104 is coated with additional substances (solid or liquid, concentrated or dispersed). In some embodiments, the sampling tip 104 may be coated by one or more materials (e.g., fluids) prior to insertion into the subject's oral cavity. In some embodiments, the sampling tip 104 is comprised of a material that is not configured to sample fluids or solids within the oral cavity to determine biological data related to the fluids or solids. For example, the sampling tip 104 may be comprised of a non-sampling substrate and may be immersed in a sampling solution prior to being inserted into the oral cavity. In other embodiments, the sampling tip 104 is comprised of a polymer or copolymer including one or more sampling chemistries (e.g., pH sampling molecules).

In some embodiments, the sampling tip 104 may include one or more capillaries configured to collect fluids from within the oral cavity through capillary action. For example, the sampling tip 104 may include one or more capillaries in fluid communication with a fluid reservoir within the sampling tip 104 where sampled fluids may be stored. In such embodiments, the portion of the sampling tip 104 proximate the fluid reservoir may be configured to provide a visual indication of the biological data relating to the sampled fluids within the fluid reservoir. In this manner, the clarity of the visual indication may be increased by concentrating a predetermined volume of sampled fluids within the fluid reservoir. For example, in instances where a pH level is being determined, storing a predetermined volume of fluids within a confined space (e.g., the fluid reservoir) may improve the clarity of the visual indication of a pH level provided by the sampling tip 104. In some embodiments, the fluid reservoir is comprised of a pH sensing material and is configured to change colors based on a pH level of the fluids contained therein.

In some embodiments, the sampling tip 104 may include a plurality of bristles extending outwardly from an outer surface of the sampling tip 104. In some embodiments, the bristles may be comprised of an adsorbent material. In some embodiments, the sampling tip 104 may have a non-planar surface area including a plurality of peaks and valleys to increase the surface area upon which fluids and/or solids may be sampled.

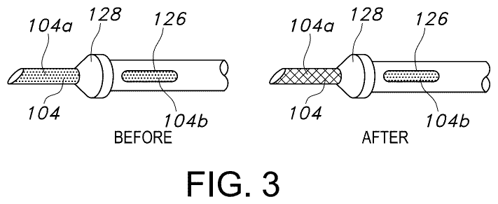

Referring to FIG. 3, in some embodiments, the oral sampling tip 104 may be configured to provide a visual indication relating to dental conditions, medical conditions, and/or a pH value of the fluids or solids located within the oral cavity. The left image of FIG. 3 is an illustration of the sampling tip 104 prior to an oral sampling, and the right image is an illustration of the sampling tip 104 during and/or following an oral sampling. For example, and as shown in FIG. 3, a distal portion 104a of the oral sampling tip 104 may be configured to provide a visual indication of, for example, a pH value of the fluids or solids within the oral cavity, that is different from a visual indication of a proximal portion 104b viewable through a window 126, as discussed in more detail below. As shown in FIG. 3, the distal portion of the oral sampling tip 104a has a first color (indicated by the dotted pattern) that is different than the proximal portion of the oral sampling tip 104b, which has a second color (indicated by the cross-hatch) different from the first. It will be understood that the patterns are intended to represent colors visible on the portions 104a-104b of the oral sampling tip and are for example only. The distal portion 104a of the sampling tip 104 may be the portion extending distally from the stopper 128 and the proximal portion 104b may be the portion that extends proximally from the stopper 128, as discussed in more detail below.

The sampling tip 104 may be configured to transition from any first color to any second color. In some embodiments, the transition in color of the sampling tip 104 may include a plurality of colors. For example, the sampling tip 104 may transition from a first color to one of a plurality of different second colors following insertion into an oral cavity of the subject. In some embodiments, the colors of the oral sampling tip 104, and the pH values they represent, correspond to the colors of conventional pH sensing agents and the respective pH values. By providing an oral sampling tip 104 configured to provide a visual indication related to biological data within the oral cavity, including for example an interproximal space thereof, the oral sampling device 100 may enable a user may easily obtain and analyze the biological data without requiring repeated visits to a dental clinic. In some embodiments, one or more of the colors of the oral sampling tip 104 may indicate the presence of dental caries, or the presence of active demineralization. For example, the visual indication (e.g., the color) of the oral sampling tip 104 may correspond to a pH value that would indicate the presence of dental caries and/or a risk of developing dental caries. In some instances the oral sampling tip 104 may enable a user to gather biological data for substantially all surfaces of the user's teeth.

The oral sampling tip 104 may be configured to detect the presence of substances within an oral cavity indicating one or more existing dental conditions and/or a risk thereof. In some embodiments, the oral sampling tip 104 may be configured to detect the presence of dentin, enamel, enamel derived and/or producing proteins such as, but not limited to, collagen and amelogenin, dentin and/or periodontal ligament degrading enzymes such as, for example, collagenase to differentiate between deep and early stage cavities. In some instances, the oral sampling tip 104 may be configured to detect the presence of catabolic enzymes such as proteases, sucrose synthase or sucrase.

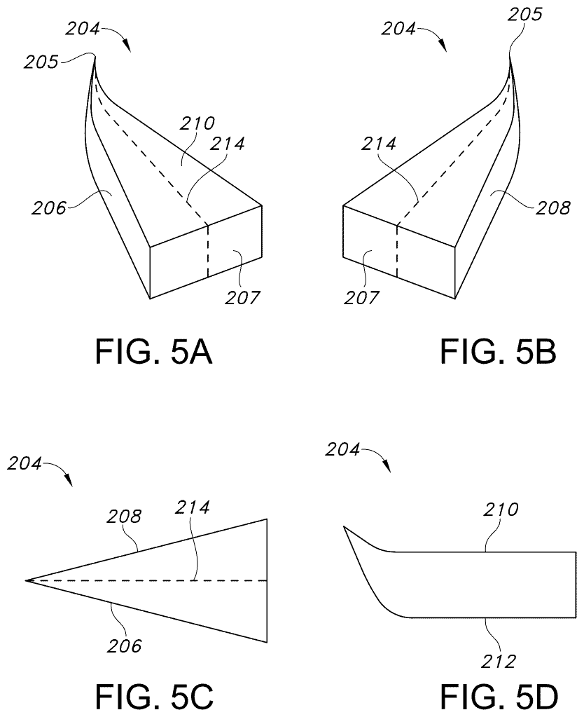

Referring to FIGS. 5A-5D, there is shown another embodiment of the oral sampling tip, generally designated 204, in accordance with an exemplary embodiment of the present disclosure. FIG. 5A is a top left perspective view of the oral sampling tip 204, FIG. 5B is top right perspective view thereof, FIG. 5C is a top elevational view thereof and FIG. 5D is a side elevational view thereof. The oral sampling tip 204 may be generally the same as the oral sampling tip 104 as discussed above, except that the oral sampling tip 204 may have a generally triangular or wedge shape. For example, the oral sampling tip 204 may include a first side surface 206 and a second side surface 208 that are each generally flat. In some embodiments, a portion of the sampling tip 204 may be configured to be inserted into an interproximal space. For example, the distal tip 205 may have a width that is thin enough to be inserted into the interproximal space such that a portion of the first side surface 206 directly contacts an interproximal side of a first tooth and the second side surface directly contacts the interproximal side of a second tooth, which is adjacent to the first tooth.

Each of the side surfaces 206, 208 may have a coating applied thereon that is configured to provide a visual indication of biological data related to the oral cavity. For example, the first side surface 206 may have a first coating applied thereon that is configured to change colors in response to a pH level of fluids or solids contained within the oral cavity. Similarly, the second side surface 208 may have a second coating applied thereon that is configured to change colors in response to a pH level of fluids or solids contained within the oral cavity. As such, the coatings applied to the first and second side surfaces 206, 208 may be comprised of pH sensing or measuring materials. In some embodiments, the first and second coatings may have a chemistry that is different from one another. For example, the first coating may be configured to transition from a first color to a second color in response to the acidity of fluids or solids of the first tooth. The second coating may be configured to transition from a third color to a fourth color in response to the acidity of fluids or solids of the second tooth. The first, second, third, and fourth colors may each be different from one another. In this manner, a user may be able to obtain separate and distinct pH measurements for the respective adjacent teeth that form the interproximal space. As such, a user may be able to easily determine, using the sampling tip 204, the presence of cavities on one or both of the adjacent teeth forming the interproximal space.

In some embodiments, the distal tip 205 of the sampling tip 204 is curved upwardly. For example, the distal tip 205 may be curved upwardly from a top surface 210 of the sampling tip 204 away from a bottom surface 212. In other embodiments, the distal tip 205 is not curved upwardly and is generally planar with the top surface 210. It will be understood that the dimensions and proportions of the sampling tip 204 shown in FIGS. 5A-5D are for illustrative purposes only and that the sampling tip 204 may have a generally wedge or triangular shape of different dimensions and/or proportions. For example, the proximal end 207 of the sampling tip 204 may have a width that is lesser than or greater than what is illustrated in FIGS. 5A-5D.

In some embodiments, the sampling tip 204 may include a delineating line 214 that extends from the distal end 205 to the proximal end 207. The delineating line 214 may provide a visual indication to the user as to where the divide between the left and right sides of the sampling tip 204 are. In some embodiments, the sampling tip 204 includes a visual indication of which side is the left and right side of the sampling tip. For example, there may be an “L” that is printed on or etched into the left side of the sampling tip 204 and an “R” that is printed on or etched into the right side of the sampling tip 204. The left and right of the sampling tip 204 referring to the areas to the left and right of the delineating line 214. In this manner, the sampling tip 204 may provide a visual indication to the user that aids the user in inserting the sampling tip 204 into the oral cavity in a desired orientation. It will be understood that the above is an example and that other visual indications may be used. It will be understood that although the sampling tip 104 is referenced throughout the following description, the sampling tip 204, as shown and described above with reference to FIGS. 5A-5D, may replace the oral sampling tip 104 that is discussed herein.

Referring back to FIGS. 1-2, the handle 102 of the oral sampling device 100 may include a wide generally flat portion configured to be easily and securely gripped between the thumb and a finger of the user and a thin elongated portion for coupling to the sampling tip 104. The generally flat portion may aid the user in maneuvering the sampling tip 104 to a desired orientation and/or position within the oral cavity. In some embodiments, the handle 102 includes a first portion 106 and a second portion 108 extending outwardly therefrom. The first portion 106 may, in some embodiments, be generally flat and/or have a width that is greater than a width of the second portion 108. For example, the first portion 106 may have a width W₁ that is greater than the width W₂ of the second portion 108. In some embodiments the width W₁ is between about two to ten times greater than the width W₂. In some embodiments, the first portion 106 may be the portion of the handle 102 configured to be gripped by the user. Put another way, the first portion 106 may provide a substantially larger area than the second portion 108 to provide the user with a larger area with which to grip the sampling device 100. The second portion 108 being substantially less wide than the first portion 106 may allow the second portion 108 to be easily inserted into, at least partially, the oral cavity of a patient and/or the user and maneuvered as desired.

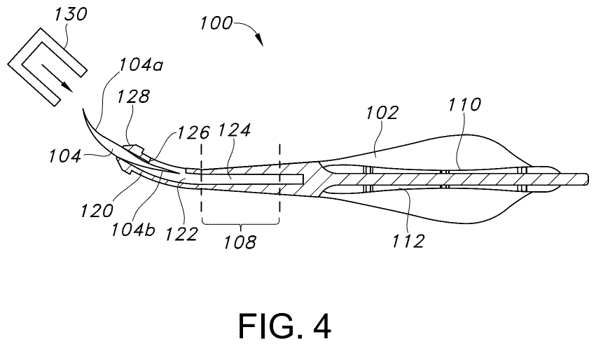

The handle 102 may include a top surface 110, a bottom surface 112 opposite the top surface 110, a first side wall 114, and a second side wall 116 opposite the first side wall 114. At least a portion of each of the first and second side walls 114, 116 may extend between the top surface 110 and the bottom surface 112. In some embodiments, each of the first and second side walls 114, 116 include a bottom portion 114a and 116a, respectively, that extends downwardly from the bottom surface 112 and away from the top surface 110. Similarly, each of the first and second side walls 114, 116 may include a top portion 114b and 116b, respectively, that extends upwardly from the top surface 110 and away from the bottom surface 112. In other embodiments, the first and second sidewalls 114, 116 may be generally flush with the top and bottom surfaces 110, 112. Put another way, in other embodiments, the first and second side walls 114, 116 do not include the respective top and bottom portions 114a-114b, 116a-116b. In some embodiments, the top surface 110 and/or bottom surface 112 have a generally concave curvature as illustrated in FIG. 4. In other embodiments, the top surface 11 and/or bottom surface 112 may be substantially parallel to one another.

In some embodiments, the oral sampling device 100 is stackable with one or more of oral sampling devices 100. For example, the bottom surface 112 may include an aperture or be generally hollow and the bottom portion 114a, 116a of the sidewalls 114, 116 may not be included. In this manner, the bottom surface 112 of a first oral sampling device 100 may be stacked on top of the top surface 110 of a second oral sampling device 100 and so on for any number of oral sampling devices 100. In some embodiments, the handle 102 may include one or more areas upon which written, or printed, information may be provided. For example, information relating to the patient, a healthcare or sampling regimen, QR code(s), and/or any other information may be printed or adhered to a surface of the handle 102. In some embodiments, the information transcribed on the handle 102 may include multiple QR codes each of which corresponding to a visual indication of the sampling tip 104 after sampling fluids or solids from within the oral cavity.

In some embodiments, at least one of the top surface 110 and bottom surface 112 include a plurality of ribbed features 118 extending at least partially along the respective top and/or bottom surface 110, 112. In some embodiments, both of the top and bottom surfaces 110, 112 include a plurality of ribbed features 118. The ribbed features 118 may be configured to reduce or at least decrease the risk of the handle 102 sliding relative to the user's hand when the user grips the handle 102, when compared to a generally smooth surface. For example, the ribbed features 118 may increase the friction between the user's fingers and/or thumb and the handle 102. In some embodiments, the ribbed features 118 extend substantially along the surface of the top surface 110 and/or bottom surface 112. In other embodiments, the handle 102 may be devoid of the ribbed features 118. In some embodiments, the ribbed features 118 may be replaced by any other means for improving the friction between the user's fingers and/or thumb and the handle 102. For example, the ribbed features 118 may be replaced by a material having a higher coefficient of friction than the material of the handle (e.g., rubber) or may include a plurality of ball like protrusions clustered together. In some embodiments, the size of the second portion 108 of the handle 102 may be altered based on an intended user. For example, the handle 102 may be custom sized to a specific user.

The handle 102 may be comprised of plastic, metal, ceramic, or mixtures thereof. In some embodiments, the handle 102 may be comprised of a bio-manufactured, biodegradable, biocompatible, recyclable, and/or edible material. In some embodiments, the handle 102 may be 3-D printed.

In some embodiments, the oral sampling device 100 includes a shaft 120 coupling the oral sampling tip 104 to the handle 102. The shaft 120 may extend outwardly from the second portion 108 of the handle 102 and is curved upwardly away from the bottom surface 112 of the handle 102. In some embodiments, the shaft 120 has a radius of curvature of between about 3 cm and 10 cm. In some embodiments, the curvature of the shaft 120 and/or a distal section of the second portion 108 of the handle 102 may aid a user in inserting the sampling tip 104 into the examined space. In some embodiments, the curvature of the shaft 120 and/or a distal section of the second portion 108 may prevent, or at least reduce, the occurrence of damage to the gingiva of the oral cavity within which the sampling tip 104 is positioned. In other embodiments, the shaft 120 may be generally flat, or straight, such that it is generally parallel with the second portion 108 of the handle 102. In other embodiments, the shaft 120 may be generally flat, or straight, and be oriented at an angle relative to the second portion 108 of the handle 102. For example, the shaft 120 may be generally straight and be oriented at an angle of between about 5 degrees and 55 degrees relative to the second portion 108. In some embodiments, the shaft 120 may be custom sized and/or shaped to a patient's mouth. In some embodiments, the shaft 120 may be 3-D printed. In some embodiments, the shaft 120 may be comprised of the same material as the handle 102.

In some embodiments, the shaft 120 is detachably coupled to the handle 102. For example, the second portion 108 of the handle 102 may include one or more mounting features (e.g., protrusions not shown) configured to receive corresponding mounting features of the shaft 120 (not shown) to allow the shaft 120 to be detachably coupled to the handle 102. In other embodiments, the shaft 120 is fixedly coupled to the handle 102. For example, the shaft 120 may be integrally formed with the handle 102. In some embodiments, the sampling tip 104 may be detachably coupled to the shaft 120. In some embodiments, the shaft 120 is comprised of a pliable material such that a user may bend or deformably adjust the shape of the shaft 120. In other embodiments, the shaft 120 is substantially rigid.

For example, and referring to FIGS. 1 and 4, the shaft 120 may define a channel 122 configured to receive the sampling tip 104. The channel 122 may extend from a distal end of the shaft 120 to a proximal end of the shaft 120 where the shaft 120 couples to the second portion 108 of the handle 102. The sampling tip 104 may extend at least partially through the channel 122 thereby coupling the sampling tip 104 to the shaft 120. In some embodiments, the second portion 108 of the handle 102 defines a connecting channel 124 configured to be in communication with the channel 122. For example, the connecting channel 124 may be generally continuous with the channel 122 such that a portion of the sampling tip 104 may extend through the channel 122 and at least partially into the connecting channel 124 of the handle 102. In some embodiments, there may be a window 126, or aperture, extending from an outer surface of the shaft 120 to the channel 122 such that a portion of the channel 122, and the portion of the sampling tip 104 disposed therein, is visible to a user. In some embodiments, the window 126 may aid the user in coupling and/or decoupling the sampling tip 104 to the shaft 120. The window 126 may also allow the user to view a visual change (e.g., color change) of the sampling tip 104 when the sampling tip 104 is used to gather biological data from within the tested space.

In other embodiments, the sampling tip 104 and shaft 120 are fixedly coupled to one another. For example, the shaft 120 may be detachable from the handle 102 and fixedly coupled to the sampling tip 104 such that a user may remove the shaft 120 and sampling tip 104 coupled thereto and replace them with a second shaft and sampling tip that are generally the same or similar. Further to this example, the sampling device 100 may be a reusable device configured to be used to obtain biological data from within the tested space more than once. For example, the handle 102 may be reusable and either the sampling tip 104 and/or shaft 120 may be single use such that a user may obtain first biological data with a first sampling tip and/or shaft. At a time following the first biological data being obtained, the sampling tip and/or shaft may be decoupled from the handle 102 and a second sampling tip and/or shaft may be coupled to the handle 102 and used to obtain second biological data from within the tested space. In other embodiments, the sampling tip 104 may be reusable. For example, the sampling tip 104 may be used to obtain first biological data, rinsed off or immersed in a neutral pH solution, and used again to obtain second biological data. In other embodiments, the shaft 120, handle 102 and sampling tip 104 are all fixedly coupled to one another and the sampling device 100 may be a single-use device that, after being used to obtain biological data within an tested space, may be discarded by the user.

In some embodiments, the shaft 120 may include a stopper 128 extending circumferentially around and outwardly from the shaft 120. The stopper 128 may be configured to limit the depth of insertion of the sampling tip 104 into the tested space. For example, the stopper 128 may have a diameter greater than the diameter of the shaft 120 such that the stopper 128 is prevented from being inserted into the tested space. In some embodiments, the stopper 128 has a minimum diameter that is greater than a gap between two adjacent teeth of the user. In some embodiments, the stopper 128 may extend only partially circumferentially around the shaft 120. In some embodiments, the stopper may include a visual indication corresponding to a visual indication of the sampling tip 104. For example, and as discussed above with reference to FIG. 3, the sampling tip 104 may be configured to provide a visual indication of biological data within the tested space. The visual indication of the sampling tip 104 may be a visual indication (e.g., a color) of a pH value of the fluids or solid and the stopper 128 may include a visual indication (e.g., a color) that corresponds to the pH value indicating the presence of dental caries. Put another way, if the pH value of the fluids or solids indicates the presence of dental caries, the color of the sampling tip 104 and the stopper 128 may be generally the same. In some embodiments, the stopper 128 may be comprised of a material different than the shaft. For example, the stopper 128 may be comprised of a rubber material.

In some embodiments, the sampling tip 104 is configured to enable partial fluid absorption and/or capillary reaction. The stopper 128 may be configured to prevent capillary action and/or absorption from occurring at the sampling tip 104 in areas proximal to the stopper 128 (e.g., portions of the stopper 128 extending proximally toward the handle 102). For example, the proximal portion 104b of the sampling tip 104 may include the portions of the sampling tip 104 positioned within the shaft 120 while the remaining portion extends distally from the stopper 128. The stopper 128 may prevent the proximal portion 104b of the sampling tip 104 from changing a visual indication in response to the sampling tip 104 being inserted into an oral cavity. For example, when inserted into the oral cavity, the distal portion 104a of the sampling tip 104 may change in color whereas the proximal portion 104a may remain the same color as it was prior to the sampling tip 104 being inserted into the oral cavity. Further to this example, the distal portion 104a may be the same or a different color than the proximal portion 104b. The proximal portion 104b of the sampling tip 104 may be viewable through the window 126. In other embodiments, the stopper 128 has no or little effect on capillary action such that both the distal and proximal portions 104a-104b of the sampling tip 104 may change in color. For example, both the distal and proximal portions 104a-104b may change from a first color to a second color in response to the sampling tip 104 being inserted into an oral cavity. In some embodiments, the strength of the capillary action of the sampling tip 104 and/or the amount of fluid sampled may cause a color change of the sampling tip to terminate distal to the window 126.

In some instances, the visual indication (e.g., color) of the distal portion 104a and proximal portion 104b of sampling tip 104 may be detectable by an image capture device operatively coupled to a computing device via an image analysis software being executed thereon.

For example, a computing device communicatively coupled to, or including an image capture device may run a color interpretation/recognition software to automatically determine the color of the distal and proximal portions 104a-104b and/or a difference in color thereof.

In some embodiments, the handle 102 may include a recess (not shown) or storage compartment that contains a plurality of sampling tips 104 and/or shafts 120. In this manner, a user may obtain first biological data, discard a first sampling tip and/or shaft and retrieve a second sampling tip and/or shaft from the recess of the handle 102 to gather second biological data. In some embodiments, one or more sampling tips 104 may be at least partially nested within one another. For example, the distal end of a sampling tip 104 may be nested within an aperture in the proximal end of an adjacent sampling tip 104. As such, a plurality of nested sampling tips 104 may be positioned within the storage compartment of the handle 102. In other embodiments, the handle 102 may include a recess (not shown) or fluid storage compartment that is configured to store a fluid for signal enhancement or reduction of the sampling tip 104. For example, the sampling tip 104 may be at least partially immersed within fluid within the storage compartment prior to and/or following insertion into the tested space to obtain biological data.

In some embodiments, the handle 102 may include one or more fluid reservoirs in fluid communication with the sampling tip 104 such that fluids stored within the handle 102 may be transferred to the sampling tip 104. For example, fluids stored within the fluid reservoir of the handle may travel through the connecting channel 124 to the channel 122 of the shaft 120 and into the sampling tip 104. In this manner, one or more sampling materials (e.g., sampling fluids), signal enhancement or reduction fluids, cleaning agents, or any other fluids may be transferred from the handle 102 to the sampling tip 104. In some embodiments, there are one or more actuators (e.g., buttons) coupled to the handle 102 and configured to selectively transfer liquids stored within the handle 102 to the sampling tip 104. For example, there may be a button coupled to the handle 102 that may be actuated by a user such that, when actuated, fluid stored within the handle 102 is free to flow through the channels 124, 122 and into the sampling tip 104. In some embodiments, one or more capsules having a predetermined amount of liquid stored therein are stored within the handle 102. The actuator may be configured to puncture or separate a first capsule from another capsule such that liquid stored within the first capsule is released and travels through the channels 124, 122 and into the sampling tip 104. In some embodiments, the actuator is a breakable dam that is configured to be easily broken by the user such that one or more fluids contained within the handle 102 may be transferred to the sampling tip 104.

In some embodiments, the sampling device 100 may include a cap 130 configured to cover the sampling tip 104. The cap 130 may be sized to substantially cover the sampling tip 104. The shape and/or size of the cap 130 may be altered based on the shape and/or size of the sampling tip 104. The cap 130 may be configured to be detachably coupled to the shaft 120 such that the cap 130 may be removed (e.g., by a user) to expose the sampling tip 104. In some embodiments, the cap 130 may be substantially transparent or translucent such that the sampling tip 104 is visible through the cap 130 when the cap 130 covers the sampling tip 104. The cap 130 may be transparent and may be comprised of a material configured to restrict wavelengths within a predetermined range from passing therethrough. For example, the cap 130 may act as a bandpass filter to restrict or enable transmission of certain wavelengths of light within a predetermined range such that the cap 130 may be coupled to the shaft 120 after the sampling tip 104 is used to sample fluids and/or solids. In this manner, the cap 130 may enhance the visibility of the visual indication present on the sampling tip 104. In some embodiments, the cap 130 may include one or more areas of visual magnification such that the sampling tip 104 appears magnified to a user viewing the sampling tip 104 through the cap 130 (e.g., when the cap 130 covers the sampling tip 104). In some embodiments, the cap 130 may contain a material to immerse the sampling tip 104 within. For example, the cap 130 may contain a sampling agent, cleaning solution, or pH neutral solution for coating the sampling tip 104. In some embodiments, the cap 130 includes one or more visual indications indicating the presence of at least one dental condition, generally the same as described above with reference to the stopper 128.

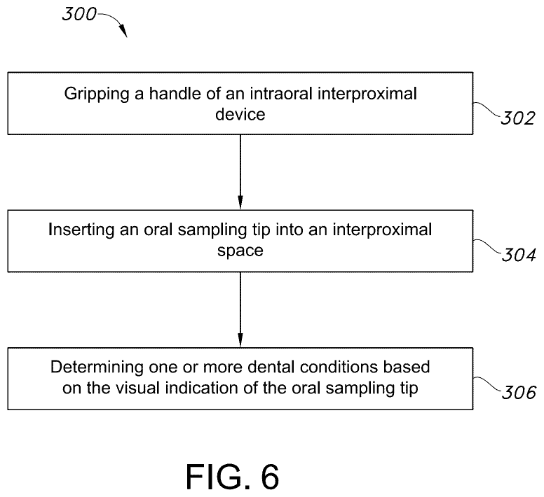

Referring to FIG. 6, there is shown a flowchart illustrating a method, generally designated 300, of using an intraoral device. Prior to the method 300, the intraoral device (e.g., the oral sampling device 100) may be removed from a packaging container. The method 300 may include the step 302 of gripping a handle of an intraoral device. For example, a user may grip the handle 102 of the intraoral sampling device 100 as shown and described above with reference to FIGS. 1-4. The method 300 may include the step 304 of inserting an oral sampling tip into an oral cavity. For example, the user may insert the oral sampling tip 104 that is coupled to the handle 102 into an oral cavity of the user. The method 300 may include the step 306 of determining one or more dental conditions based on the visual indication of the oral sampling tip. For example, the oral sampling tip 104 may sample fluids or solids within the oral cavity to determine biological data of said fluids or solids. The oral sampling tip 104 may provide a visual indication, as described above, such as a visual color change that may be used to determine the presence, or lack thereof, of one or more dental conditions, such as, but not limited to dental caries. For example, the visual color change of the oral sampling tip may result in a second color that corresponds to a pH value that indicates the presence of dental caries. In some instances, the method 300 may include repeating step 306 for a portion of, or substantially, all surfaces of the teeth of a user to determine dental conditions thereof. For example, the oral sampling tip 104 may be used to gather biological data from each surface of each tooth of a subject thereby providing an accurate determination of where dental caries is present with regards to the subject's teeth.

In some embodiments, inserting the oral sampling tip into the oral cavity includes inserting the oral sampling tip into an interproximal space. For example, the oral sampling tip 104, or sampling tip 204 may be inserted into an interproximal space in the oral cavity. A pH value of under about 5.5 may indicate that at least one of the teeth that defines the interproximal space is experiencing mineral loss. As such, the user may determine one or more dental conditions of the interproximal tooth surface based on the change, of the sampling tip 104, from a first color to a second color. For example, the second color may be used to determine a measure of the acidity of the fluids or solids located on the other tooth within the interproximal space.

In some embodiments, the method 300 may include detaching the oral sampling tip from the handle, coupling a second oral sampling tip to the handle, inserting the second oral sampling tip into the tested space in the oral cavity, and obtaining, via the second oral sampling tip, second biological data of the fluids or solids located within the tested space. For example, as mentioned above, the sampling tip 104 and/or the shaft 120 may be detachable from the handle 102. As such, a user may obtain first biological data using a first sampling tip and then decouple the first sampling tip from the handle 102. The user may then couple a second sampling tip to the handle 102 and insert the second sampling tip into the same tested space to gather second biological data.

In some embodiments, the sampling tip 104 may be configured to clean the user's teeth prior to being used to obtain biological data. For example, there may be a first tip generally similar to sampling tip 104 except that it may be generally rigid such that it may be used to remove debris from within or around the tested space (e.g., similar to a toothpick). In other embodiments, the first tip may include one or more non-adsorbent fibers to allow for the delivery of one or more pharmacological agents and/or dental materials useful for cleaning the tested space. As such, a user may attach the first tip to the handle 102, in generally the same manner as the sampling tip 104, clean within or around the tested space, and then attach the sampling tip 104 to obtain biological data. In some embodiments, one end of the sampling tip 104 (e.g., the proximal end) includes means for obtaining biological data (e.g., a pH sampling tip) and the remaining end (e.g., the distal end) includes means for cleaning within or around the tested area. In this manner, a user may clean within and/or around the tested space and may remove the sampling tip 104 from the handle 102, rotate it 180 degrees, reattach the sampling tip 104 to the handle 102 and obtain biological data.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

1. An oral sampling device comprising:
a handle configured to be gripped by a user; and
an oral sampling tip extending outwardly from the handle, the oral sampling tip sized to be inserted into an oral cavity of the user and provide a visual indication of biological data for fluids or solids located within the oral cavity.

2. The oral sampling device of claim 1, wherein the handle includes a first portion and a second portion, the first portion having a width that is greater than a width of the second portion and the oral sampling tip is coupled to the second portion.

3. The oral sampling device of claim 1, wherein the oral sampling tip is detachably coupled to the handle.

4. The oral sampling device of claim 2, wherein a shaft extends outwardly from the second portion of the handle and is curved upwardly away from a bottom surface of the handle.

5. The oral sampling device of claim 4 further comprising:
a stopper coupled to a distal end of the shaft, the stopper having a diameter that is greater than a diameter of the shaft, the stopper configured to prevent a visual indication of a portion of the sampling tip extending proximally toward the handle from changing.

6. The oral sampling device of claim 5, wherein the visual indication is representative of a pH value of the fluids or solids located within the oral cavity.

7. The oral sampling device of claim 6, wherein the stopper is colored to match the visual indication of the oral sampling tip when the pH value of the fluids or solids within the oral cavity indicate a presence of dental caries.

8. The oral sampling device of claim 1, wherein the oral sampling tip includes a pointed distal tip.

9. The oral sampling device of claim 1, wherein the oral sampling tip is comprised of an adsorbent material.

10. The oral sampling device of claim 1, wherein the oral sampling tip is comprised of paper.

11. The oral sampling device of claim 1, wherein the oral sampling tip has a maximum thickness of between about 0.025 mm to about 3.000 mm.

12. The oral sampling device of claim 1, wherein the handle includes a top surface and bottom surface each having a generally concave curvature.

13. The oral sampling device of claim 12, wherein the handle includes first and second side walls opposite one another and each extending between the bottom surface and the top surface, each of the first and second side walls includes a bottom portion extending or not extending downwardly from the bottom surface away from the top surface and a top portion extending upwardly from the top surface away from the bottom surface.

14. The oral sampling device of claim 12, wherein at least one of the top surface and bottom surface include a plurality of ribbed features extending along the respective top surface and bottom surface.

15. The oral sampling device of claim 1, wherein the oral sampling tip is configured to be inserted into one or more of a pit of a tooth, a fissure in a tooth, and an interproximal space in the oral cavity.

16. A method of using an intraoral device, the method comprising:
gripping a handle of the intraoral device;
inserting an oral sampling tip coupled to the handle into an oral cavity of a user; and
determining one or more dental conditions of the oral cavity based on an indication generated by the oral sampling tip relating to biological data of fluids or solids located within the oral cavity.

17. The method of claim 16 further comprising:
detaching the oral sampling tip from the handle;
coupling a second oral sampling tip to the handle;
inserting the second oral sampling tip into the oral cavity of the user; and
obtaining, via the second oral sampling tip, second biological data of the fluids or solids located within the oral cavity.

18. The method of claim 16, wherein there is a shaft coupling the oral sampling tip to the handle, the shaft being detachably coupled to the handle and the oral sampling tip is fixedly coupled to the shaft, the method further comprising:
detaching the shaft and oral sampling tip fixedly coupled thereto from the handle;
coupling a second shaft having a second oral sampling tip fixedly coupled thereto to the handle;
inserting the second oral sampling tip into the oral cavity of the user; and
obtaining, via the second oral sampling tip, second biological data of the fluids or solids located within the oral cavity.

19. The method of claim 16, wherein inserting the oral sampling tip into the oral cavity causes the oral sampling tip to change from a first color to a second color, and
wherein determining one or more dental conditions of the oral cavity includes determining, based on the change from the first color to the second color, a measure of acidity of the fluids or solids located within the oral cavity.

20. The method of claim 16, wherein inserting the oral sampling tip into the oral cavity includes inserting the oral sampling tip into one of: a pit of a tooth, a fissure in a tooth or an interproximal space in the oral cavity.

21. A cavity detection device comprising:
a handle having a first portion and a second portion, the first portion having a width that is greater than a width of the second portion;
a shaft coupled to and extending outwardly from the second portion of the handle, the shaft being curved away from a bottom surface of the handle;
a stopper coupled to a distal end of the shaft, the stopper having a diameter that is greater than a diameter of the shaft; and
a plurality of oral sampling tips each configured to be detachably coupled to the handle, and each being configured to be inserted into an oral cavity of a user to measure a pH value within the oral cavity and change from a first color to a second color to provide a visual indication of the measured pH value to indicate a presence of dental caries.
